(19) United States
(12) Patent Application Publication
(10) Pub. No.: US 2009/0209456 A1
(43) Pub. Date: Aug. 20, 2009

(54) COMPOSITIONS AND METHODS FOR IMPROVING FACIAL AND BODY AESTHETICS

(76) Inventor: Iliana Sweis, Highland Park, IL (US)

Correspondence Address: K&L Gates LLP, P.O. Box 1135, CHICAGO, IL, 60690, US

(21) Appl. No.: 12/033,460

(22) Filed: Feb. 19, 2008

(51) Int. Cl. A61K 38/13; A61K 31/395; A61K 31/728; A61K 38/39; A61K 31/19; A61Q 19/08

(52) U.S. Cl. 514/9; 514/183; 514/54; 514/12; 514/557

(57) ABSTRACT

Compositions and methods of treating facial and body tissue aesthetics and reconstruction are disclosed using immunosuppressive agents either by themselves or in conjunction with other soft tissue fillers.

### BACKGROUND

Cosmetic plastic surgery is gaining a greater acceptance in society as a whole. The emphasis on youth and beauty in our culture has fueled significant recent innovations in cosmetic surgery, procedures, especially non-invasive (non-surgical) procedures. For example, there were over 11.5 million cosmetic procedures performed in the United States in 2006. Of these, 1.92 million were surgical procedures (17%) and 9.53 million were non-surgical procedures (83%). Between 1997 and 2006, there was a tremendous increase in the number of cosmetic procedures performed in the United States. For women, there was a 123% increase in surgical procedures and a 749% increase in non-surgical procedures, whereas for men, there was a 2% decrease in surgical procedures and a 722% increase in non-surgical procedures.

Attempting to revise the effects of aging, for example, is driving a significant amount of the increase in cosmetic procedures. Data from the U.S. Government Administration on Aging indicate that Americans are living and working longer than in the past. According to the data, the number of Americans who live beyond their 65th birthday over the next two decades has increased by 38%. Between 2000 and 2030, the number of persons 65 years of age or older is expected to more than double. Combating the cosmetic changes that occur with aging is an important priority for adult Americans of any age. In 2006, Americans spent nearly $12.2 billion for cosmetic medically-based procedures: $7.6 billion for surgical procedures and $4.5 billion for non-surgical procedures.

Aesthetic procedures are no longer limited to the older segment of the population, however. Data from the American Society for Aesthetic Plastic Surgery breaking down the procedures by age group indicate that a significant number of younger patients are utilizing these services. In fact, nearly half of all cosmetic procedures are performed on those between 35 to 50 years of age. Persons within this age group represent an important and growing element of the population. The emphasis on youth and appearance will continue to support and stimulate cosmetic procedures to combat the changes associated with aging.

Aging involves specific predictable physiologic and structural changes that occur throughout an individual's life. Age-associated changes in the skin include: static and dynamic wrinkles, overall thinning of the skin, benign growths, alterations induced by ultraviolet irradiation (photoaging), loss of volume, bone resorption, and decreased skin elasticity and hydration. Aging of the facial soft tissues, in particular, can be identified clinically by a number of changes. These include, for example, loss of skin elasticity, thinning of the dermis, atrophy of fat, greater visibility of bony landmarks, blood vessels, mimetic and non-mimetic facial wrinkles, furrows, lowering of the eyebrows, lowering of the cheek fat pad, ptosis of the nasal tip, descent of the corners of the mouth, formation of jowls, and sagging of the neck. Clinical alterations in the lower face due to aging occur mostly due to loss of volume in the lower third of the face and include prominence of the nasolabial folds, ptosis of the oral commissures, thinning of the lips (red lip), flattening and lengthening of the upper lip (white lip), atrophy of Cupid's bow, and deepening of the prejowl sulcus.

The etiology of these changes is multifactorial and includes such causes as gravity, sun damage, environmental pollutants, atrophy and redistribution of fat, damage to proteins such as collagen and elastin, loss of hyaluronic acid, damage to genes of epithelial and connective tissues, and repetitive activity of muscles.

Fillers have been used to repair, restore or augment hard or soft tissue contour defects of the body due to aging, injury, as well as acquired or congenital deformities of the face, body and internal organs. Fillers are natural or synthetic substances that have been used to reduce wrinkles, restore lost volume, hydrate the skin, soften nasolabial folds, augment and contour lips, improve scars (depressed, hypertrophic and keloid scars), strengthen weakened vocal cords, and provide other soft tissue improvements. Substances that have been utilized include fat, paraffin, Gore-Tex®, human collagen, bovine collagen, silicone, and hyaluronic acids. In 1981, a new era in soft tissue fillers emerged with the FDA approval of bovine collagen. Since then, many new soft tissue fillers have emerged. The dramatic increase in the number of current and investigational fillers has been fueled by many factors including improvements in biotechnology, constant and increasing emphasis on cosmesis by our society, as well as an unmet need to match the dramatic improvements produced by botulinum toxin type A in the upper facial regions. With the introduction of these newer fillers, there has been an ongoing need to evaluate their risk/benefit profiles and define their limitations in order to maximize patient cosmetic outcomes and safety.

The most widely used products available worldwide today are human and bovine collagen, hyaluronic acids, polymethylmethacrylate (PMMA), calcium hydroxyappetite and autologous fat. To date, however, the ideal filler does not exist. The currently available tissue fillers are transient in nature necessitating frequent treatments. There is therefore a need for compositions and methods for the treatment of facial and body tissue aesthetics and reconstruction.

### SUMMARY

The present disclosure relates to compositions and methods of improving facial and body tissues. In particular, the present disclosure relates to the use of immunosuppressive agents such as calcineurin inhibitors either alone or in combination with soft tissue fillers to cosmetically improve features or treat defects of soft tissues of the face and body.

Accordingly, the present disclosure provides a composition comprising an effective amount of at least one immunosuppressive agent and an effective amount of at least one soft tissue filler.

In an embodiment, the immunosuppressive agent is a calcineurin inhibitor.

In an embodiment, the calcineurin inhibitor is selected from the group consisting of a cyclosporin, tacrolimus and pimecrolimus.

In an embodiment, the effective amount of cyclosporin is from about 1 mg to about 500 mg (per dose).

In an embodiment, the effective amount of tacrolimus is from about 0.5 mg to about 5 mg (per dose).

In an embodiment, the effective amount of pimecrolimus is from about 1 mg to about 5 mg (per dose).

In an embodiment, the soft tissue filler is selected from the group consisting of fat, Gore-Tex®, hyaluronic acids, polymethylmethacrylate, calcium hydroxyappetite, human collagen, bovine collagen, fascia, poly-L-lactic acid, and any future soft tissue fillers, or combinations thereof.

In an embodiment, the soft tissue filler is derived from a source selected from the group consisting of a human source, an animal source, a bacterial source, a synthetic source, an embryonic tissue source and a stem cell source.

In an embodiment, the composition is in a form selected from the group consisting of an oral form, a sublingual form, a topical form, a transdermal form, a submucosal form, and an injectable form an extended release form, a sustained release form, an immediate release form and combinations thereof.

In an embodiment, the composition includes an effective amount of a mammalian target of Rapamycin inhibitor.

Another embodiment of the present disclosure provides a method of treating soft tissue of an individual comprising administering the composition to the individual.

A further embodiment of the present disclosure provides a method of treating soft tissue comprising administering to an individual in need of improved soft tissue a composition including an effective amount of at least one immunosuppressive agent.

In an embodiment, the method includes administering the composition systemically.

In an embodiment, the method includes locally injecting the composition into an area of the body of the individual selected from the group consisting of dermal tissue, subdermal tissue, subcutaneous tissue, intramuscular tissue, supra-periosteal tissue, sub-periosteal tissue and intravascular.

In an embodiment, the composition is administered to treat at least one of the conditions selected from the group consisting of thin lips, wrinkles, folds, glabellar lines, nasolabial folds, mouth angle folds, nasal or chin ptosis, decreased skin tone, scarring, facial and body soft tissue defects and volume deficiency.

In an embodiment, the condition is present in the face or body of the individual.

It is therefore an advantage of the present disclosure to provide compositions and methods that are effective in causing the body to use its own mechanisms to improve soft tissue in the body.

Another advantage of the present disclosure includes improving and prolonging the effects of soft tissue fillers.

A further advantage of the present disclosure includes reducing the cost of improving soft tissues of the face and body.

Additional features and advantages are described herein, and will be apparent from, the following Detailed Description.

### DETAILED DESCRIPTION

The present disclosure relates to compositions and methods of improving the contour, shape or texture of facial and body tissues. In particular, the present disclosure provides compositions and methods for improving the tissues by administering one or more immunosuppressive agents such as a calcineurin inhibitor to the tissue either as monotherapy or as a supplement to injection of soft tissue fillers. The present disclosure is directed to the use of immunosuppressive agents to repair, replace or augment hard or soft tissues of the face and body, such as skin, tendon, cartilage, bone, muscle, interstitium and other connective tissue.

Immunosuppressive agents are known to decrease an immune response and have been used to control the immune response to foreign antigens in the treatment of transplant organ rejection, as well as to control the immune response to self-antigens in the treatment of autoimmune disease. Immunosuppressive agents and, in particular, calcineurin inhibitors such as cyclosporin and tacrolimus, have been shown to decrease inflammation while increasing fibroblast proliferation and fibrogenesis. All current uses of immunosuppressive agents rely on their anti-inflammatory properties, and the increase in fibroblast proliferation and fibrogenesis is seen as an undesirable side effect following long-term systemic exposure to these drugs. This mechanism is thought to be responsible for the renal fibrosis associated with prolonged cyclosporin systemic exposure in renal transplant patients. It is believed that immunosuppressive agents increase fibroblast proliferation and fibrogenesis by increasing local levels of transforming growth factor-beta 1 (TGF-β1). TGF-β1 has been shown to be fibrogenic through its ability to promote extracellular matrix accumulation and fibroblast proliferation. TGF-β1 is also a cytokine that plays a role in tissue repair and remodeling.

Although all current uses of immunosuppressive agents rely on their anti-inflammatory, properties, it has been surprisingly found by the inventor, however, that immunosuppressive agents may actually increase the volume of tissue in an area by increasing the local production of skin constituents. Furthermore, it has been found by the inventor that patients exposed to immunosuppressive agents, in particular calcineurin inhibitors, have prolonged longevity of injected cosmetic soft tissue fillers. Due to their effect on TGF-B1 or any of the other isoforms of TGF-B, increasing fibrogenesis or through a yet undefined mechanism, immunosuppressive agents may modulate local or systemic collagen levels, elastin levels, hyaluronic acid levels, ground matrix levels, fibroblast levels or other constituents of the skin. In addition, through a yet undefined mechanism, immunosuppressive agents enhance the effect and longevity of cosmetic soft tissue fillers. This may be due to their effect in modulating the local or systemic production of skin constituents or due to increasing the local tissue tolerance to the injected soft tissue filler. As such and without being bound to any particular hypothesis or theory, immunosuppressive agents may be used to cosmetically improve or to treat defects of the soft tissues and potentially boney structures of the face and body.

In an embodiment of the present disclosure, a composition is provided. The composition includes an effective amount of an immunosuppressive agent. As referred to herein, an immunosuppressive agent may include any substance that decreases a function of an immune system through any mechanism. The immunosuppressive agent may interfere with the ability of the immune system to respond to antigenic stimulation by inhibiting cellular and humoral immunity. For example, an immunosuppressive agent may include a substance that decreases the production of antibodies through mechanisms such as suppressing the activity of the lymphocytes that form antibodies. An immunosuppressive agent may include any substance that suppresses cell-mediated immunity through, for example, reducing T cell proliferation. An immunosuppressive agent may include any substance that decreases cytokine synthesis through, for example, inhibition of genes that code for cytokines such as IL-1, IL-2, IL-3, IL-4, IL-5, IL-6, IL-8 and TNF-γ, any substance that suppress humoral immunity through decreased expression of IL-2 and of IL-2 receptors by B cells to reduce B cell clone expansion and antibody synthesis, any substance that affects the proliferation of both T cells and B cells, substances that prevent the clonal expansion of lymphocytes during an immune response, any substance that interferes with the synthesis of nucleic acids and any substance that increases the expression or changes the function of certain adhesion molecules (α4/β7 integrin) in lymphocytes to cause the lymphocytes to accumulate in the lymphatic tissue thereby decreasing their number in the circulation. In particular, an immunosuppressive agent may include any suitable immunosuppressive agent that affects the production of collagen, hyaluronic acid, elastin, ground matrix, fibroblasts and other constituents in the skin, soft tissues or related structures. It should be appreciated that the immunosuppressive agent of the present disclosure may include any suitable combination of immunosuppressive agents involving any number of the same or different mechanisms.

In an embodiment, the immunosuppressive agent includes an effective amount of a calcineurin inhibitor such as cyclosporin, pimecrolimus and tacrolimus (FK506). Calcineurin inhibitors inhibit calcineurin which is a protein phosphatase that induces different transcription factors such as calcineurin dephosphorylates (NFATc) that are important in the transcription of IL-2 genes when activated. Calcineurin is activated when intracellular concentration of calcium increases in a T-helper cell when the T-cell receptor interacts with an antigen. IL-2 activates T-helper lymphocytes and induces the production of other cytokines to control the action of cytotoxic lymphocytes and NK cells. The amount of IL-2 being produced by the T-helper cells is believed to influence the extent of the immune response significantly. Calcineurin inhibitors, therefore, reduce transcription of IL-2 to prevent activation of T-cells and to inhibit production of other cytokines resulting in a decreased immune response.

Other immunosuppressive agents may include anti-proliferatives such as azathioprine nitrogen mustard, cyclophosphamide, chloramphenicol, actinomycin, colchicines and mycophenolic acid, corticosteroids such as prednisolone and hydrocortisone, or small biological agents such as FTY720. An immunosuppressive agent may also include glucocorticoids, cytostatics, alkylating agents such as nitrogen mustards (cyclophosphamide), nitrosoureas, platinum compounds, antimetabolites and folic acid analogs such as methotrexate, purine analogs such as azathioprine and mercaptopurine, pyrimidine analogs and protein synthesis inhibitors, cytotoxic antibiotics such as dactinomycin, anthracyclines, mitomycin C, bleomycin, and mithramycin.

As referred to herein, an effective amount of the immunosuppressive agent may include the minimum effective concentration of the immunosuppressive agent as determined by standard pharmacologic testing. It should be appreciated that the effective amount may vary depending upon whether the agent is administered locally or systemically. In an embodiment the systemic dose is lower than the local dose. Treatment with the immunosuppressive agent may be repeated as needed, using long-term local exposure or short-term systemic exposure. Treatment may be required repeatedly for a number of days, and may be repeated at various time intervals depending upon the need to treat the soft tissues. The introduction of these agents into the tissues is not limited to any particular modality. They may be used in oral form, sublingual form, topical form, transdermal form, submucosal form, injection form (dermal, subdermal, subcutaneous, intramuscular, supra-periosteal, sub-periosteal or intravenous) and as extended, sustained or immediate-release form.

Examples of an effective amount of the immunosuppressive agent for cyclosporin may include from about 1 mg to about 500 mg administered in concentrations from about 25 μg/ml to approximately 10,000 μg/ml. In an embodiment, the effective amount of cyclosporin may include a local injection dose from about 1 mg to about 100 mg, or a local injection dose from about 100 mg to about 200 mg, or a local injection dose from about 200 mg to about 500 mg. In an embodiment, the effective amount of cyclosporin may include an oral dose from about 50 mg to about 250 mg, or an oral dose from about 250 mg to about 500 mg to achieve approximate systemic levels of 50 mg to 300 mg per dose.

An effective amount of the immunosuppressive agent tacrolimus may include from about 0.5 mg to about 5 mg administered in concentrations from about 2 μg/ml to approximately 200 μg/ml. In an embodiment, the effective amount of tacrolimus may include a local injection dose from about 0.5 mg to about 1 mg or a local injection dose from about 1.5 mg to about 2 mg, or a local injection dose from about 2.5 mg to about 5 mg, or a local injection dose from about 5 mg to about 10 mg. In an embodiment, the effective amount of tacrolimus may include an oral dose from about 0.5 mg to about 2.5 mg or an oral dose from about 0.5 mg to about 5.0 mg to achieve approximate systemic levels of 2 mg to 10 mg per dose.

An effective amount of the immunosuppressive agent pimecrolimus may include from about 1 mg to about 5 mg administered in concentrations of from about 1 mg/ml to approximately 100 mg/ml. In an embodiment, the effective amount of pimecrolimus may include a topical dose from about 1 mg to about 2.5 mg or a topical dose from about 2.5 mg to about 5 mg.

In an embodiment, an agent is administered to control the growth or expansion of collagen, hyaluronic acid, elastin, ground matrix, fibroblasts or other skin constituents accompanying the use of immunosuppressive agents. In an embodiment, Mammalian Target of Rapamycin (mTOR) inhibitors such as Rapamycin (sirolimus) or everolimus mall be administered to inhibit the increase in formation of collagen, hyaluronic acid, elastin, ground matrix, fibroblasts or other skin constituents accompanying the use of immunosuppressive agents as presently disclosed.

An effective amount of the mTOR inhibitor may include the minimum effective concentration of the mTOR inhibitor as determined by standard pharmacologic testing. For example, an effective amount of the mTOR inhibitor sirolimus may be from about 1 mg to about 10 mg, and an effective amount of the mTOR inhibitor everolimus may be from about 1 mg to about 5 mg. It should be appreciated that the mTOR inhibitor may be administered in any suitable concentration to enable determination of the effect of the mTOR inhibitor on the improvement of the tissue before administering a subsequent dose of immunosuppressive agent or mTOR inhibitor.

Treatment with the mTOR inhibitors may be repeated as needed, using long-term local exposure or short-term systemic exposure. Treatment may be required repeatedly for a number of days, and may be repeated at various time intervals depending upon the need to treat the soft tissues. The introduction of these agents into the tissues is not limited to any, particular modality. They may be used in oral form, sublingual form, topical form, transdermal form, submucosal form, injection form (dermal, subdermal, subcutaneous, intramuscular, supra-periosteal, sub-periosteal or intravenous) and as extended, sustained or immediate-release form.

The composition of the present disclosure may include any pharmaceutically acceptable excipients suitable for the particular formulation of the composition Excipients may include aqueous or nonaqueous vehicles, buffers, suspending and dispensing agents, colorants, flavors, binders, starches or sugars, disintegrating agents, lubricants, glidants and any suitable combination thereof. The composition may be formulated as an extended, sustained or immediate release form.

In an embodiment, the composition is administered to an individual in need of improved soft or hard tissue. The compositions of the present disclosure may be used to repair, restore or augment hard or soft tissue contour defects of the body due to aging or injury as well as acquired or congenital deformities of the face, body and internal organs. For example, the compositions of the present disclosure may be used to treat conditions involving thin lips, facial wrinkles and folds including glabellar lines, nasolabial folds, mouth angle folds. The compositions of the present disclosure may also be used for lip augmentation or recontouring; improvement in jowls and neck contour; correction of nasal or chin ptosis; restoring volume around boney landmarks of the face and body; improving wrinkles (rhytids) and skin tone, texture and thickness along the neck, chest, breasts, arms, hands, torso, legs, and feet. Further, the compositions of the present disclosure may be used to improve scars including depressed, hypertrophic and keloid scars or in fresh incisions to improve the resulting scars. The compositions of the present disclosure may also be used to improve the effect of soft tissue fillers implanted in the face or any other area of the body including internal organs, bones or structures. For example, the compositions of the present disclosure may be used to strengthen weakened or paralyzed vocal cords. It should be appreciated that the condition present in the face or in the body of the individual may be cosmetic or functional in nature.

The composition may be formulated as an extended, sustained or immediate release form. The composition ma)y be formulated to be administered as an oral form, a topical form, a transdermal form, a submucosal form, and an injectable form.

The composition may be administered to an individual by any suitable modality or route of administration. The composition may be administered systemically such as intravenously or orally. The composition may be administered locally such as at the site of the defect or at an alternative site or sites. The composition may be administered by injection into dermal tissue, subdermal tissue, subcutaneous tissue, intramuscular tissue, supra-periosteal tissue, sub-periosteal tissue or combinations thereof.

Treatment may be required repeatedly for a number of days, and may be repeated at various time intervals depending upon the soft tissue needs. The methods of the present disclosure may include varied dosing schedules for introducing the calcineurin inhibitors and other immunosuppressive agents into the skin. In an embodiment, an effective amount of the immunosuppressive agent is administered in multiple doses. The doses may increase or decrease over time. For example, in an embodiment, a relatively high loading dose may be administered followed by subsequent administration of smaller doses. Administration of the immunosuppressive agent may be repeated as needed. It should be appreciated that the methods of the present disclosure may include the administration of the composition at any suitable frequency and levels of dosing sufficient to optimize the effect of the immunosuppressive agent on local collagen levels, elastin levels, hyaluronic acid levels, ground matrix levels, fibroblast levels or other tissue constituents.

The administration of the immunosuppressive agent may, be short term or, if used locally, long term. Administration of the immunosuppressive agent may include a single dose administered once or multiple doses over multiple treatments. For example, in a particular embodiment, local administration of an immunosuppressive agents may include as many as 30 doses administered over 30 days. In an embodiment, 10 doses of the composition are administered over a period of 10 days. In an embodiment, 5 doses of the composition are administered over a period of 5 days. In an embodiment, oral administration of an immunosuppressive agent may include less than or more than about 5 doses administered over about one month.

Following an initial procedure or treatment, the present disclosure may include an evaluation of the effect of the treatment and a determination of whether one or more touch-up or follow-up treatments may be performed. The composition may be administered subsequent to the initial treatment at any suitable period of time after the initial procedure and at any suitable frequency. For example, in an embodiment, if the result of the injection after a suitable period of time such as a week or month is estimated to be less than about 80% correction, a second touch-up treatment may be administered during a subsequent week or month. It should be appreciated that administration of an effective amount of the composition followed by an evaluation of its effect may be repeated as many times as necessary to achieve a desired outcome in the treatment.

In an embodiment, the action of the immunosuppressive agent is modulated by light-based therapies such as phototherapy, laser, or any other suitable light-based therapy.

In an embodiment, an immunosuppressive agent is administered to an individual in combination with one or more soft tissue fillers. The immunosuppressive agent may be administered before administration of the soft tissue filler, together with the soft tissue filler, following administration of the soft tissue filler or combinations thereof. For example, in various embodiments, the immunosuppressive agent may be administered about one day before the administration of a soft tissue filler, within about one hour after the administration of a soft tissue filler, about one day after the administration of a soft tissue filler, about seven days after the administration of a soft tissue filler, and combinations thereof. It should be appreciated that any suitable temporal relation between the administration of the immunosuppressive agent and the administration of the soft tissue filler may be employed according to the methods of the present disclosure.

Immunosuppressive agents may also be used in combination with soft tissue fillers to enhance the effects of the immunosuppressive agents or to prolong the beneficial effects of soft tissue fillers. In addition to the improvement of tissue volume and integrity by immunosuppressive agents, alone, the increased efficacy and stability of the soft tissue fillers by immunosuppressive agents may be due to the property of immunosuppressive agents to suppress the immune system reactions to the soft tissue fillers thereby creating tolerance to the soft tissue fillers.

In an embodiment of the present disclosure, the composition includes an effective amount of a soft tissue filler. Soft tissue fillers include materials that are safe and suitable for implantation into an individual while producing minimal side effects. Soft tissue fillers may include any suitable material or combination of materials that are substantially non-immunogenic, non-carcinogenic, non-teratogenic, non-infectious, non-migratory, physiologic, hypoallergenic, easy to inject, effective in producing natural-looking results that mimic those produced by, the body's own connective tissue and ground substance, effective in correcting a variety of facial and body wrinkles and folds, acquired or congenital facial and body defects and combinations thereof In addition, a suitable soft tissue filler may require special equipment such as syringes and local anesthesia to administer.

Soft tissue fillers may be permanent or non-permanent in nature. Permanent fillers are considered to include any filler that is substantially non-degradable and typically remains within the tissues for a period greater than 12 months. Non-permanent fillers are considered to include fillers that are biodegradable and remain in the tissues from about 3 to about 12 months depending on the metabolism of the individual, the nature of the filler and the location into which the filler is injected. In an embodiment, the soft tissue filler is long-lasting once administered but not permanent. Soft tissue fillers may be removed by the body's own mechanisms, preferably while maintaining a reasonable cosmetic effect during this process. However, the cosmetic effect may dissipate requiring further administration of the filler. The filler may or may not be removable or reversible if required.

The soft tissue fillers of the present disclosure may be derived from a variety of sources. These sources may include animal, non-animal, such as bacterial, and synthetic sources. In particular, the soft tissue fillers may be derived from a human source, animal source, bacterial source, synthetic source, embryonic tissue source, stem cell source, or any other source that may serve as a tissue filler itself or enhance tissue fillers. Soft tissue fillers may be autogenic, allogenic or xenogenic to the individual receiving the soft tissue filler.

For example, the soft tissue fillers of the present disclosure may, include autologous fat, bovine or human collagen, non-animal stabilized hyaluronic acid (NASHA), Restylane®, Perlane®, Restylane Fine Line®, Juvederm Ultra®, Juvederm Ultra Plus®, Hylaform®, Hylaform Plus®, Sculptra®, Fascian® and Radiesse®, any combination thereof, and any future soft tissue fillers.

The soft tissue fillers of the present disclosure may include any suitable concentration or individual constituent particle size. The soft tissue fillers may or may not be cross-linked and may be cross-linked using any suitable type of cross-linking agent and any suitable amount of cross-linking agent, such as glutaradehyde and the like. The soft tissue fillers of the present disclosure may include any suitable gel viscosity, and may, be monophasic or biphasic. The soft tissue fillers of the present disclosure may be administered with any suitable anesthetic agent. For example, an anesthetic such as 5% Lidocaine may be applied topically or introduced by injection prior to or included with an injection of the soft tissue filler.

The soft tissue filler of the present disclosure may include collagen. Collagen is a fibrous, extracellular, insoluble protein comprising a major component of connective tissues throughout the animal kingdom. Type I collagen is the most common subtype of collagen in normal skin. In an embodiment, the soft tissue filler includes an injectable form of collagen.

The injectable collagen may include varying concentrations of highly purified human collagen or non-human collagen, such as bovine collagen. Human-based collagens may be obtained from cadavers or human fibroblastic cell culture grown in a controlled laboratory environment. Biochemical processing or cross-linking may be employed to reduce the antigenicity or the rate of proteolytic cleavage of the collagen molecule. Bovine collagen may be obtained from the skin of cattle. These herds may be isolated and carefully controlled against contact with other animals, thus reducing the risk of viral or prion contamination. Bovine collagen may also be obtained from cattle embryonic tissues.

In an embodiment, an effective amount of an immunosuppressive agent is administered to an individual receiving a soft tissue filler including bovine collagen to, among other purposes, reduce the need for collagen test injections and to reduce an immunologic reaction to bovine collagen. An estimated 3% to 10% of patients develop an immunologic reaction to bovine collagen; however, a much smaller number actually manifests a clinically relevant response. As a result of this immunologic reaction, administration of a soft tissue filler including bovine collagen may require one or more negative collagen test injections prior to its use. After a first test injection is performed (usually in the forearm), the area is monitored for a period of about 4 weeks. If there is no reaction such as redness, pruritis, swelling or inflammation, one or more additional tests injection may be performed and also followed for about 4 weeks to ensure lack of an immune response. Because of the need for a skin test preventing immediate treatment of an individual, bovine collagen is no longer used as often as other fillers. However, administration of an effective amount of an immunosuppressive agent according to the present disclosure may reduce the frequency and extent to which an immunologic reaction associated with using soft tissue fillers including bovine collagen occurs.

The soft tissue filler of the present disclosure may include hyaluronan. Hyaluronan, also called hyaluronic acid, is a component of all connective tissues and is abundant within the human dermis. Hyaluronic acid is a natural complex sugar (glycosaminoglycan biopolymer) found throughout all living organisms. In particular, the hyaluronic acid molecule is a polysaccharide with a uniform, linear, non-branching structure. It is composed of repeating disaccharide units: N-acetylglucosamine and D-glucuronic acid linked by alternating beta-1,4 and beta-1,3 glycosidic bonds. The soft tissue filler of the present disclosure may include any suitable number of disaccharide units and, the molecular weight of such hyaluronic acid may vary greatly depending on the source, location and function. It should be appreciated that the heavier molecular weight hyaluronic acids are best suited for deeper injections while the lighter molecular weight hyaluronic acids are better suited for superficial injections. The chemical structure of hyaluronic acid is uniform throughout all living species and is a component of the extracellular space of virtually all vertebrate species, thus there is minimal chance of immunogenicity associated with including hyaluronan derived from an allogeneic source in the soft tissue fillers of the present disclosure.

Hyaluronic acid is a beneficial component of a soft tissue filler of the present disclosure. Hyaluronic acid has the ability to maintain the structure and function of tissues by creating volume, lubricating tissue, and modulating cell integrity, mobility, and proliferation. Hyaluronic acid creates volume in tissues by forming a random coil in tissue that holds water and allows passage of metabolites to and from cells. Hyaluronic acid lubricates tissues by stabilizing intracellular structures by creating an elastoviscous matrix. Hyaluronic acid modulates cell integrity, mobility, and proliferation by assisting in cell differentiation, migration, and wound repair. As a highly permeable compound, hyaluronic acid regulates transport of solutions and cell movement and function and aids in wound healing.

Hyaluronic acid is an important component of the extracellular space and, in particular, the extracellular space surrounding cells of the skin where more than half of the total body hyaluronic acid in an individual is present. The polar nature of the sugar residues in the polysaccharide makes the hyaluronic acid molecule highly hydrophilic. The hydrophilic nature of hyaluronic acid attracts and maintains water within the extracellular space. When in solution, the extensive length of the hyaluronic acid molecule allows the molecule to twist, bend, and intertwine to form a unique three-dimensional coil. Its hydrophilic nature combined with its three-dimensional structure allows it to bind or absorb more than 1000 times its own weight in water. In doing so, hyaluronic acid hydrates, lubricates, and stabilizes connective tissue, thus allowing cell movement and tissue remodeling and consequently adding volume to the skin.

In higher concentrations, coils of hyaluronic acid molecules entangle to form a network of macromolecules that entrap large amounts of water and provide structural support while facilitating the passage of nutrients, cytokines, and metabolites to and from cells. Hyaluronic acid provides a fluid matrix or lattice on which collagen and elastic fibers may develop. Hyaluronic acid binds with collagen and elastin and transports essential nutrients to these fibers. The triple combination of collagen, elastin, and hyaluronic acid provides structure, elasticity, and volume to the skin and contributes to its overall appearance. The ability, of hyaluronic acid to bind with water creates volume in the skin and allows the skin to remain hydrated and supple. In addition to being highly hydrophilic, hyaluronic acid also maintains the viscoelasticity of the skin. Without hyaluronic acid, the skin would appear dry, withered, and wrinkled.

Such a soft tissue filler may be administered to individuals who, for example, suffer from an inability to produce adequate amounts of hyaluronic acid due to aging skin, exposure to environmental pollutants and ultraviolet rays, and other causes of decreased production of hyaluronic acid. An inability to produce adequate amounts of hyaluronic acid may cause the skin to begin to lose hydration and volume, resulting in the formation of wrinkles and folds. The physiology, amount, and compartmentalization of cutaneous hyaluronic acid change with age. These changes not only alter the hydration of the skin, but also decrease its elasticity making it more susceptible to injure and infection.

In an embodiment, the soft tissue filler includes soft tissue fillers comprised of crossed-linked hyaluronic acid. In its natural form, hyaluronic acid forms a liquid made of highly hydrated individual polymers that are completely metabolized in the body within 24 hours. Since unmodified hyaluronic acid is rapidly cleared by the body, approximately one third of the body's hyaluronic acid is degraded and replaced daily. Turnover occurs through a process that begins with unraveling of hyaluronic acid molecules. After the individual hyaluronic acid molecules disentangle themselves from the macromolecular network, they bind to cell membrane receptors, undergo endocytosis, and are degraded within lysosomes to their basic constituents.

With such a rapid turnover, the hyaluronic acid molecule may be modified to extend its half-life. This modification may include stabilizing the molecule through a process of cross-linking in which the individual chains of hyaluronic acid are chemically bound (cross-linked) together, transforming the liquid hyaluronic acid into a soft solid, or gel. The firmness of the gel depends on the degree of cross-linking of the individual hyaluronic acid chains. The greater the cross-linking the firmer the gel. In order to metabolize the cross-linked hyaluronic acid, the body must break down all of the cross-links and the individual polymers. This allows slower metabolism of the cross-linked hyaluronic acids, allowing a longer duration of effect when they are used therapeutically.

Although modification of hyaluronic acid molecules increases tissue residency, viscosity, and elasticity, in an embodiment, extensive modification is avoided to preserve biocompatibility maintain its hydrophilic properties, limit the potential for immunogenicity or immunologic reactivity, limit the potential for molecule migration, maintain a particle size that does not interfere with the dynamics of the injection process, and maintain a substantially soft gel consistency of the hyaluronic acid. In an embodiment, the modification maintains or preserves the basic molecular structure of the molecule.

In an embodiment, the soft tissue filler comprising the crossed-linked hyaluronic acid is injected into the skin to replace lost hyaluronic acid and to temporarily restore skin volume. In an embodiment, the soft tissue filler includes injectable hyaluronic acid gels. Injectable hyaluronic acid gels may be non-immunologic, highly elastoviscous, hydrating, and have beneficial space-occupying properties. The hyaluronic acid gels may be from bacterial sources (Non-animal Stabilized Hyaluronic Acids) or extracted from rooster combs. Cross-links between the hydroxyl groups of the individual molecules may be introduced to render the molecule less biodegradable following injection. The resultant polymer may be insoluble, resistant to migration, and have greater elasticity than native hyaluronan.

Hyaluronic derivatives are safe, practical to use, and eliminate the need for allergy testing. Hylaform®, Restylane and Juvederm® products impart a natural texture and appearance to the skin. Overcorrection is not required, and the products are relatively straightforward to use by a trained physician. Although some of these products last longer than collagen, they are not permanent.

suitable soft tissue filler of the present disclosure includes Hylaform®. Hylaform® is a hyaluronic acid of animal origin. In an embodiment, hyaluronic acid is extracted from the combs of roosters after the combs are finely minced and mixed with a solvent. Despite a vigorous manufacturing process, Hylaform® may, contain minute amounts of blood, cellular, extracellular impurities, and potentially bacterial and fungal contaminants. Hylaform® may be stabilized through cross-linking in one or more stages. In an embodiment, even, fifth disaccharide unit of Hylaform® is cross-linked with glutaraldehyde in a first stage and cross-linked with vinyl sulfone in a second stage.

The total degree of cross-linking of hyaluronic acid in Hylaform® is approximately 20%, which reduces the biocompatibility of the product. Reduced biocompatibility can increase degradation of the product and increase its antigenicity. However, other forms of hyaluronic acid, such as Restylane®, require minimal modifications during the stabilization process and, therefore, do not significantly alter the purity, biochemical properties, or antigenicity, and retain their biocompatibility throughout the degradative process.

The soft tissue filler of the present disclosure may include materials such as Restylane®. In an embodiment, Restylane® may be used for the treatment of mid-to deep-dermal implantation for the correction of moderate to severe facial wrinkles and folds, such as the nasolabial folds. Restylane® is available in three forms: Restylane®; Restylane Fine Lines®; and Perlane®. All Restylane® preparations have a hyaluronic acid concentration of 20 mg/mL; however, the three preparations vary in the constituent particle size. Restylane Fine Lines®, Restylane®, and Perlane® have particle sizes of about 150 mm, about 250 mm, and about 1000 mm, respectively. In an embodiment, smaller particle size is used for upper dermal injections, whereas the larger particles have increased lifting capacity and are used for mid- to deep-dermal areas.

The soft tissue fillers of the present disclosure may include materials that undergo isovolemic degradation. The metabolism of hyaluronic acids, for example, involves isovolemic degradation. According to this principle, as the hyaluronic acid is broken down, the remaining hyaluronic acid molecules have an increased ability to bind with water over time. This enables hyaluronic acids to retain greater volume for longer periods, thereby maintaining correction longer than bovine collagen products. Isovolemic degradation preserves the filler's aesthetic effect and provides substantially constant soft tissue correction over time without retarding the natural degradative process of the body. For example, Restylane® and Juvederm® provide substantially constant soft tissue correction over time due to principles of isovolemic degradation.

Soft tissue fillers comprising hyaluronic acid such as Restylane®, for example, may be removed from the tissues by a combination of events. Initially, the soft tissue filler may undergo endocytosis in the cells at the injection site. Once intracellular, the molecules are broken down and released for lymphatic uptake. They then undergo partial depolymerization and are delivered into the blood stream. Blood is then cleared of the hyaluronic acid by its degradation to CO₂ and H₂O.

The soft tissue fillers of the present disclosure may include the hyaluronic acid, Juvederm® manufactured by Allergan, Inc. Juvederm® comes in three different preparations including: Juvederm 30®, Juvederm 24HV® (Juvederm Ultra), and Juvederm 30HV® (Juvederm Ultra Plus®). Juvederm® consists of cross-linked hyaluronic acid produced by Streptococcus equi bacteria and suspended in a physiologic buffer. Juvederm® may be formulated to a hyaluronic acid concentration of 24 or 30 mg/mL.

Unlike other currently approved hyaluronic acid dermal fillers, Juvederm® does not utilize a gel particle suspension formulation. Juvederm® uses Hylacross Technology®, which results in a smooth gel formulation. The gel particle technology used in Restylane® can be visibly seen as opposed to the smoother formulation used in Juvederm®. The Juvederm® formulation allows smooth flow and extrusion, resulting in less pain on injection and less tissue trauma than associated with Restylane®. Juvederm® may be used for injection into the mid to deep dermis for correction of moderate to severe facial wrinkles and folds (such as nasolabial folds). Resylane® and Juvederm® have the advantage of containing no animal components and both have been subjected to more extensive clinical evaluation than Hylaform®.

In an embodiment of the present disclosure, a botulinum toxin, such as botulinum toxin type A (Botox Cosmetic®), may be administered to an individual in need of improved soft tissue. The botulinum toxin may be administered before, after or together with at least one immunosuppressive agent or at least one soft tissue filler. The botulinum toxin may be included in a composition comprising at least one immunosuppressive agent, at least one soft tissue filler or combinations thereof. Using botulinum toxin may increase the longevity of a soft tissue filler by altering the local soft tissue environment into which it is injected. For example, Botox Cosmetic® may relax the muscles that contribute to wrinkles to enhance the results of soft tissue fillers used to soften existing lines and folds. Botox Cosmetic® may also reduce repetitive activity that underlies the formation of rhytides, adjunctive treatment before soft-tissue augmentation (with collagen, hyaluronic acid, PMMA, and other soft tissue fillers) which may naturally extend the duration of effect and prevent the reformation of wrinkles.

In an embodiment, soft tissue fillers are implanted intradermally by methods such as injection. Prior to the injection of any soft tissue fillers, the individual may be questioned about bleeding disorders as well as previous historic of bleeding following individual procedures or accidents. In addition, the individual may be advised to avoid drugs that can significantly interfere with platelet function within about a week of the procedure.

Soft tissue fillers of the present disclosure, such as collagen, hyaluronic acid fillers, Radiesse, and others may be injected by any suitable techniques that may vary With the physician's experience and preference, and patient characteristics. Any of the soft tissue fillers may be injected within the dermis subdermis, subcutaneous tissues, muscular tissues, and supra-periosteal or sub-periosteal level. In general, the soft tissue filler may be provided in a sterile syringe or sterile container that may be approved for one-time usage or multi-usage. The syringe or container may be of any suitable volume and size. An anesthetic such as 5% Lidocaine may be applied topically or injected locally prior to the injection.

Injection techniques may include any suitable technique such as serial puncture, linear threading, serial threading, fanning and cross-hatching. Serial puncture involves multiple, closely spaced injections along fine lines, wrinkles, or folds. Although serial puncture allows precise placement of the filler, this technique produces multiple puncture wounds that may be undesirable to some patients. Linear threading is accomplished by full), inserting the needle into the middle of the wrinkle or fold and injecting the filler along the track as a “thread.” Although threading is most commonly practiced after the needle has been fully inserted and is being withdrawn, it can also be performed while advancing the needle (“push-ahead” technique). Serial threading is a technique that utilizes elements of both serial puncture and linear threading approaches.

Fanning involves the same needle placement as does linear threading; however, the needle direction malt be continually changed in a clockwise or counter clockwise direction without fully withdrawing the needle. Fanning minimizes the number of puncture sites in patients with large treatment regions. Cross-hatching consists of a series of parallel linear threads injected at intervals of about 5 to about 10 mm followed by a new series of threads injected at right angles to the first set to form a grid. This technique is particularly useful in facial contouring when coverage of the treatment region needs to be maximized.

In an embodiment, the lips may also be treated to enhance the definition of the lip line or to increase the volume of the lips themselves. To create a more pronounced definition of the lip line, fillers may be injected along the vermillion border. Redefining this edge leads to a more youthful and appealing look. The philtrum columns and cupid's bow may also be better defined through this technique. To add volume to the lips, fillers may, be placed along the vermillion border and within the red lip dry mucosa.

The compositions of the present disclosure may also be administered by a transdermal device. Accordingly, topical administration may be accomplished using a patch either of the reservoir or porous membrane type or of a solid matrix variety.

The physician may treat each area to full correction. Full correction may be assessed to mean that the treated area was at level with the surrounding skin with elimination of the fold or wrinkle. In the case of lips, full correction may be considered when the desired volume is achieved. In addition, any changes in the feeling, texture, and color of the treated area may be considered in determining an effective amount.

Assessment of outcomes may be evaluated at various time periods such as 2, 4, and 6 months using any suitable method. For example, assessment of outcomes may be evaluated using the investigator-based Wrinkle Severity Rating Scale (WSRS) and Global Aesthetic Improvement Scale (GAIS). The WSRS is a photograph-based evaluation method designed to quantify the severity of facial folds. Based on the length and apparent depth of the fold, the severity is scored on a 5 point photographic scale (rated as absent, mild, moderate, severe, or extreme). The GAIS, unlike the WSRS, is a relative scale; that is, the investigator grades the overall improvement in appearance on a 5-item scale (“worse” to “very much improved”) by comparing the patient's appearance with a pretreatment high-magnification photograph.

The following prospective examples are illustrative of determining the effective amount as referred to in the present disclosure but are not intended to limit the present disclosure in any way.

### EXAMPLES

The initial study to evaluate the potential use of calcineurin inhibitors, cyclosporin and tacrolimus in modulating the production of collagen, hyaluronic acid, elastin, ground matrix, fibroblasts and other skin constituents will be conducted using the rabbit model. The rabbits will be divided in to three primary groups, the cyclosporin group, the tacrolimus group, and the control group. The skin of the rabbits in the control group will be injected only with the hyaluronic acid, Juvederm®. The skin of the rabbits in the cyclosporin group will be injected at specified sites either with cyclosporin alone or cyclosporin and Juvederm® combination. The skin of the rabbits in the tacrolimus group will be injected at specified sites either with tacrolimus alone or tacrolimus and Juvederm® combination. Both groups of calcineurin inhibitors will be further divided into three groups based on the dosing frequency, of the immunosuppressive agent injections: The first group will receive a single injection; the second group will receive 5 injections over 5 days, the third group still receive 10 injections over 10 days. The single injection group will receive its calcineurin inhibitor injection 12 to 24 hours after the Juvederm® injection. The other two groups receiving multiple calcineurin inhibitor injections will receive the first of these 12 to 24 hours after the Juvederm® injection. All Juvederm® and calcineurin inhibitor injections will be placed in the deep dermis of the skin along the back of the rabbit.

The back of each rabbit will be shaved and divided into a grid of 3 bilateral regions for a total of 6 regions. Each grid will allow a treatment area of 1×1 cm. Cyclosporin (rabbit equivalent of 2-10 mg/kg/day) or tacrolimus (rabbit equivalent of 0.1 mg/kg/day) will be injected into the 3 grids on one side of the back at 3 different dosing intervals: Single injection, 5 injections over 5 days, and 10 injections over 10 days. Simultaneously, on the contralateral side, the same calcineurin inhibitor using the same dosing schedule will be injected following a one-time injection of Juvederm®. The control group will receive a single Juvederm® injection only. The treated areas along the back skin will then be harvested at up to 3 months following the last injection of the calcineurin inhibitor. These areas will be evaluated for levels, type and morphology of collagen, hyaluronic acid, elastin, ground matrix, fibroblasts, TGF-β1, TGF-β2, TGF-β3, and connective tissue growth factor. The results will be used to compare the effect of calcineurin inhibitor monotherapy and the effect of calcineurin inhibitor and hyaluronic acid combination therapy to the effect of hyaluronic acid monotherapy. These evaluations and levels will be assessed using immunohistochemical analysis, quantitative polymerase chain reaction, and electron microscopy.

It should be appreciated that other dosage forms such as oral (systemic) dosing of immunosuppressive agents may be used to determine the resulting soft tissue correction. It should be further appreciated that similar protocols may be used to determine the minimum effective dose for other immunosuppressive agents contemplated in the present disclosure. The effects of mTOR inhibitors will be similarly studied in the rabbit model.

Once the effects of the immunosuppressants on soft tissue augmentation are noted in the animal model and safety is ascertained using toxicity studies, human studies will be undertaken. As an additional safety measure, all of the initial studies will be first carried out in the forearms of healthy volunteers and once the cosmetic effect is ascertained, the studies will be conducted along the nasolabial folds. The initial studies will consist of three groups. The first group will undergo placement of Juvederm® alone along one nasolabial fold and placement of Juvederm® with a calcineurin inhibitor along the contralateral nasolabial fold. The second group will undergo placement of Juvederm® alone along one nasolabial fold and placement of the calcineurin inhibitor alone in the contralateral nasolabial fold. The third group will undergo placement of Juvederm® alone along both nasolabial folds. The most appropriate dosing schedule of the injection of the calcineurin inhibitor will be determined from the results of the animal study. The local injection of the calcineurin inhibitor will be given to achieve the same exposure and concentration as safe systemic dosing. The local concentration of cyclosporin will not exceed 1000 μg/ml of blood or tissue and the local concentration of tacrolimus will not exceed 20 μg/ml of blood or tissue. These are small doses and considered quite safe. These patients will then be evaluated in a blinded fashion by an independent investigator to determine any local reactions as well as the longevity and degree of correction (adequate correction, insufficient correction, or excessive correction). These evaluations will be conducted at 2 and 4 weeks, then at 3, 6 12 and 24 months using the investigator-based Wrinkle Severity Rating Scale (WSRS) and Global Aesthetic Improvement Scale (GAIS).

In the case of excessive correction, the mTOR inhibitors will then be used to evaluate their potential to minimize the changes that occur with the various calcineurin inhibitors.

It should be understood that various changes and modifications to the presently preferred embodiments described herein will be apparent to those skilled in the art. Such changes and modifications can be made without departing from the spirit and scope of the present subject matter and Without diminishing its intended advantages. It is therefore intended that such changes and modifications be covered by the appended claims.

The invention is claimed as follows:

1. A composition comprising an effective amount of at least one immunosuppressive agent and an effective amount of at least one soft tissue filler.

2. The composition of claim 1, wherein the immunosuppressive agent is a calcineurin inhibitor.

3. The composition of claim 2, wherein the calcineurin inhibitor is selected from the group consisting of a cyclosporin, tacrolimus and pimecrolimus.

4. The composition of claim 1, wherein the soft tissue filler is selected from the group consisting of fat, Gore-Tex®, hyaluronic acids, polymethylmethacrylate, calcium hydroxyappetite, human collagen, bovine collagen, fascia and poly-L-lactic acid.

5. The composition of claim 4, wherein the soft tissue filler is derived from a source selected from the group consisting of a human source, an animal source, a bacterial source, a synthetic source, an embryonic tissue source and a stem cell source.

6. The composition of claim 1, which is in a form selected from the group consisting of an oral form, a sublingual form, a topical form, a transdermal form, a submucosal form, an injectable form, an extended form, a sustained release form, an immediate release form and combinations thereof.

7. The composition of claim 1, which includes an effective amount of a mammalian target of Rapamycin inhibitor.

8. A method of treating soft tissue of an individual comprising injecting a comprising an effective amount of at least one immunosuppressive agent and an effective amount of at least one soft tissue filler in an individual in need of same.

9. A method of treating soft tissue comprising administering a composition comprising an effective amount of at least one immunosuppressive agent to an individual in need thereof.

10. The method of claim 9, wherein the immunosuppressive agent is a calcineurin inhibitor.

11. The method of claim 10, wherein the calcineurin inhibitor is selected from the group consisting of cyclosporin, tacrolimus and pimecrolimus.

12. (canceled)

13. The method of claim 9, comprising a soft tissue filler derived from a source selected from the group consisting of a human source, an animal source, a bacterial source, a synthetic source, an embryonic tissue source and a stem cell source.

14. The method of claim 9, wherein the composition is formulated in a form selected from the group consisting of an oral form, a sublingual form, a topical form, a transdermal form, a submucosal form, an injectable form, an extended form, a sustained release form, an immediate release form and combinations thereof.

15. The method of claim 9, which includes administering the composition systemically.

16. The method of claim 9, which includes injecting the composition into an area of the body of the individual selected from the group consisting of dermal tissue, subdermal tissue, subcutaneous tissue, intramuscular tissue, supra-periosteal tissue, sub-periosteal tissue and intravascular.

17. (canceled)

18. The method of claim 9, wherein the composition is administered to treat at least one of the conditions selected from the group consisting of thin lips, facial and bodes wrinkles and folds, glabellar lines, nasolabial folds, mouth angle folds, nasal or chin ptosis, decreased skin tone, scarring, soft tissue defects and volume deficiency.

19. The method of claim 18, wherein the condition is present on the face or body of the individual.
